(19) United States
(12) Patent Application Publication
(10) Pub. No.: US 2007/0031611 A1
(43) Pub. Date: Feb. 8, 2007

(54) Ultrasound medical stent coating method and device

(76) Inventor: Eilaz P. Babaev, Minnetonka, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/197,915

(22) Filed: Aug. 4, 2005

(51) Int. Cl. A61L 33/00; B05D 3/00; B06B 1/00; B05C 5/00

(52) U.S. Cl. 427/600; 427/2.1; 118/300

(57) ABSTRACT

An ultrasound apparatus and technique produces precise and uniform coatings on various substrates such as stents or other medical devices. The apparatus and technique increases adhesiveness of the surface of the stent or other medical device. In addition, the coating, drying, sterilization processes take place concurrently. The apparatuses generate and deliver targeted, gentle, and highly controllable dispensation of continuous liquid spray. The ultrasound coating apparatuses and techniques provide an instant on-off coating process with no atmospheric therpeutic agent contamination, no “webbing,” no “stringing” or other surface coating anomalies. Furthermore, the technology reduces wastage of expensive pharmaceuticals or other expensive coating materials.

### BACKGROUND OF THE INVENTION

1. Field of the Invention

The present invention relates to coating technologies, and more particularly, to an apparatus and a method of using ultrasound energy for coating the surfaces of various types of medical devices such as stents, catheters, implants, etc.

2. Description of the Related Art

Human and animal blood vessels and other cavities and lumens are commonly treated by mechanically enhancing blood flow through expanding the damaged wall area with stents, which are implantable mesh tub devices. Stents generally can be divided into two categories: metallic bar stents and therpeutic agent eluting stents. The therpeutic agent eluting stents are coated with a polymer and therpeutic agent to reduce adverse physiological reactions, such as restenosis, etc.

Due to specific construction and design of stents and insufficient existing coating technologies and methodologies, it has been extremely difficult to coat the inner and outer surface of stents uniformly and/or evenly. Moreover, issues also exist with respect to coating repeatability without webbing or stringing and controlling the dosage of therpeutic agent-polymer coating. In some instances, a release profile of a therpeutic agent can be optimized by varying coating thickness along the surface of the medical device. For example, the coating thickness may be varied along the longitudinal axis of a stent by increasing the thickness of the coating at the end section of the stent as compared to the middle portion in order to reduce risk of restenosis caused by the stent's end sections.

Coatings have been applied to the surface of stents and other medical devices on both the interior and exterior of the device both by different techniques such as mechanical coating, gas spray coating, dipping, polarized coating, electrical charge (electrostatic) coating, ultrasound coating, etc. Coatings have been applied by combinations of dipping and spraying. Ultrasound energy or ultrasound spraying have also been used for applying coatings, as has dipping the stent in an ultrasonic bath.

All of the coating technologies and methods existing to date have critical shortcomings. Such shortcomings include non-uniformity of coating thickness, webbing, stringing, bare spots on the surface, therpeutic agent wasting, over spray, difficulties with control of therpeutic agent flow volume, and adhesivity problems. Current coating technologies also require a long drying time and subsequent sterilization. Therefore, there is a need for a method and device for defect-free, controllable coating technologies and methods for stents and other medical devices.

FIGS. 1, 2, and 3 show most close prior arts to present invention—ultrasonic sprayer in use with the cone spray pattern according to U.S. Pat. No. 6,569,099. According to prior art, liquid drop or flow 2 from tube 9 being delivered directly to the radial surface 5 or radiation surface 6 of the ultrasonic tip 1, which creates the spray 3 and delivers to the wound 4.

FIGS. 4 and 5 shows drawbacks of prior art, in this case, portion 7 of liquid 2 is being dripped from the radial surface 5 or radiation surface 6 and being wasted without getting sprayed. Additionally, dripping of liquid creates turbulence and non-uniformity of spay, which causes non-uniformed coating layer. Dripping results in excessive waste of expensive therpeutic agents and changing the uniformity of the spray particles which prevents even coating of the stent. Furthermore, spray pattern of the prior art is cone and the cross section of the spray pattern is rounded, which is does not match to the stent configuration. This is an important distinction because such pattern oversprays the stent surface which results once more in therpeutic agent waste and inability to control the thickness of the coating layer. The prior arts method and device can be successfully used in wound treatment because of the cheap price of saline and other antibiotics and relatively big size of treatment area. The prior art device cannot be used in stent coating because of very expensive therpeutic agents for stent coating and high demand for quality such as uniformity and control of coating layer.

Therpeutic agents, polymers, their combination or mixtures do not easily wet the stent surfaces, and it is difficult to achieve easy contact between the coating and the stent surface. Furthermore, therpeutic agent+polymer mixture reduces wettability of stents from different materials such as: 316-L, 316-LS stainless steel, MP-35 alloy, nitinol, tantalum, ceramic, aluminum, titanium, nickel, niobium, gold, polymeric materials, and their combination. Wettability or adhesivity can be increased by different methods, such as: primer coating, etching by chemicals, exposing the stent surface to electrical corona (ionization of air around electrical conductors), plasma, etc., but surface energy from such methods dissipates quickly, limiting the time when stent should be coated. Primer coating such as urethane, silicons, epoxies, acrilates, polyesters need to be very thin and compatible with the therpeutic agent, polymer or their mixtures are applied on top of it.

### SUMMARY OF THE INVENTION

The present invention is directed toward apparatus and methods for defect-free, controllable coating technologies and methods applicable to stents and to other medical devices. The present invention, an ultrasonic method and device for stent coating, will provide a controllable coating thickness without webbing and stringing. The thickness of the coating may be changed along the axis of the stent or other medical device.

According to the most general aspect of the invention, a controlled amount of liquid is delivered to the distal end of an oscillating member—ultrasonic tip with the rectangular shape to create rectangular pattern of fine spray. Liquid may be delivered via precise syringe pumps or by capillary and/or gravitational action. In this case, the amount of delivered liquid must be approximately the same volume or weight of coating layer and must be determined experimentally.

The distal end of the liquid delivery tube/vessel must be rectangular or flat which should match the geometrical shape of ultrasonic tips distal end to create even and uniformed flat or elongated spray pattern.

Ultrasonic sprayers typically operate by passing liquid through the central orifice of the tip of an ultrasound instrument. A gas stream delivers aerosol particles to the surface being coated. Currently, no ultrasound stent coating application without the use of gas/air stream delivery with the precise control of delivered liquid volume has been indicated. Several problems occur.

First, rounded spray pattern/cone cannot deliver therpeutic agent directly to the stent surface without waste of the expensive therpeutic agent.

Second, minimum diameter of liquid particles in the 40 to 60 micron range cannot coat the stent with a 5-30 micron coating thickness.

Furthermore, the drip of the liquid from the radiation surface results in the waste of the expensive therpeutic agent and changes the uniformity of the coating layer.

The proposed technique for coating medical devices and stents, includes creation of a spray pattern, which matches the geometrical shape of stents or surface to be coated. The technique also consists of using a number of acoustic effects of low frequency ultrasonic waves. These acoustic effects have never been used in coating technology. In addition, the technique includes spinning the stent and moving the ultrasound coating head during the coating process to create special ultrasonic—acoustic effects, which will be described in detail below. All coating operations are controlled by special software program to achieve high quality results.

The proposed method can coat rigid, flexible, and self expanded stents made of different materials, such as metals, memory shape alloys, plastics, biological tissues and other biocompatible materials.

The volume of coating liquid starts from 1 micro liter and increases with very precise control of spray delivery process with 100% delivery.

The technique may also include directing additional gas flow into the coating area. Gas flow may be hot or cold and directed through the particle spray or separate from the particle spray.

The apparatus consists of ultrasonic tips specifically fabricated to avoid the waste of spray liquid and allow control of the spraying process. The rate of ultrasound frequency may be in the range between 20 KHz and 200 KHz or more. The preferable ultrasound frequency is in the range of 20-60 KHz, with a recommended frequency of 60 KHz. Under robotic control, each tabletop device can coat, dry, and sterilize 60 to 100 stents per hour or more depending upon the requested thickness of the coating layer.

Thereby, the proposed apparatus and method for ultrasound stent coating results in uniform, even, controllable and precise therpeutic agent or polymer delivery with no webbing, stringing. Furthermore, coating, drying and sterilization of coating layer occur simultaneously with the increased adhesivity properties of stent surface.

One aspect of the invention may provide an improved methods and devices for coating of medical implants such as stents.

Another aspect of this invention may provide a methods and devices for drug and polymer coating of stents using ultrasound.

Another aspect of this invention may provide methods and devices for coating stents, that provides controllable thickness of coating layer.

Another aspect of the invention may provide method sand devices for coating of stents that provides changeable thickness of coating layer along the longitudinal axis of the structure.

Another aspect of the invention may provide methods and devices for coating of stents that avoid the coating defects like webbing, stringing, and the like.

Another aspect of the invention may provide methods and devices for coating of stents, which increases the adhesivity property of stents along the longitudinal axis of the structure with no chemicals.

Another aspect of the invention may provide methods and devices for coating of stents, that provides drying of coating layer along the longitudinal axis of the structure simultaneously with the coating process.

Another aspect of the invention may provide methods and devices for coating of stents, that provides sterilization of coating layer along the longitudinal axis of the structure simultaneously with the coating process.

### BRIEF DESCRIPTION OF THE DRAWINGS

The present invention will be shown and described with reference to the drawings of preferred embodiments and will be clearly understood in details.

FIG. 1 is a cross sectional view of an ultrasonic sprayer in use with the cone spray pattern in currently available devices;

FIG. 2 illustrates the delivery of liquid directly to radiation surface of ultrasonic tip according in currently available devices;

FIG. 3 illustrates the delivery of liquid directly to radial surface of ultrasonic tip according in currently available devices;

FIG. 4 is a cross sectional view of ultrasonic sprayer in currently available devices that shows the dripping of liquid from radial or radiation surface of the ultrasonic tip;

FIG. 5 is a three-dimensional view of the ultrasonic sprayer with the cone spray pattern in currently available devices with the dripping of liquid from radial or radiation surface of ultrasonic tip;

FIG. 6 is a cross sectional view of an ultrasonic sprayer tip with landing space for liquid drops or flow in use with the flat (from upside) spray pattern according to concept of present invention;

FIG. 7 is a three dimensional view of an ultrasonic sprayer tip with landing space for liquid drops or flow in use with the flat (from upside) spray pattern according to concept of present invention;

FIG. 8 is a cross sectional view of an ultrasonic sprayer tip with landing space for liquid drops or flow in use and cut from down part of tip (with the flat from upside and downside spray pattern) according to concept of the present apparatus;

FIG. 9 is a three dimensional view of an ultrasonic sprayer tip with landing space for liquid drops or flow in use and cut from down part of tip (with the flat from upside and downside spray pattern) according to concept of the present apparatus;

FIG. 10 is a three dimensional view of an ultrasonic sprayer tip with landing space for liquid drops or flow in use and rectangular form of radiation surface to create rectangular or flat spray without dripping according to concept of the present apparatus;

FIG. 11 is a three dimensional view of an rectangular ultrasonic sprayer tip with landing space for liquid drops in one point via liquid delivery tub/vessel in use and rectangular form of radiation surface to create rectangular or flat spray without dripping according to concept of the present apparatus;

FIG. 12 is a three dimensional view of a rectangular ultrasonic sprayer tip with landing space for liquid drops via multiple tub/vessels in width of cross section in use and rectangular form of radiation surface to create rectangular or flat spray without dripping according to concept of the present apparatus, and also shows the spinning stent on a spindle or mandrel;

FIG. 13 is a three dimensional view of an rectangular ultrasonic sprayer tip with landing space for liquid flow in width of cross section in use and rectangular form of radiation surface to create rectangular or flat spray without dripping according to concept of the present apparatus wherein the liquid delivery tube/vessel's cross-section is as rectangular as the ultrasonic tip's distal end or radiation surface;

FIG. 14 is an illustration of acoustic effects of part of ultrasound stent coating process with no spray;

FIG. 15 is an illustration of acoustic effects of ultrasound stent coating process with spray;

FIG. 16 is a three dimensional illustration of ultrasonic tip with the specific construction of distal end for stent coating; and,

FIG. 17 is a cross sectional view of an ultrasonic sprayer with the axial orifice in use with the rectangular/flat spray pattern according to present invention.

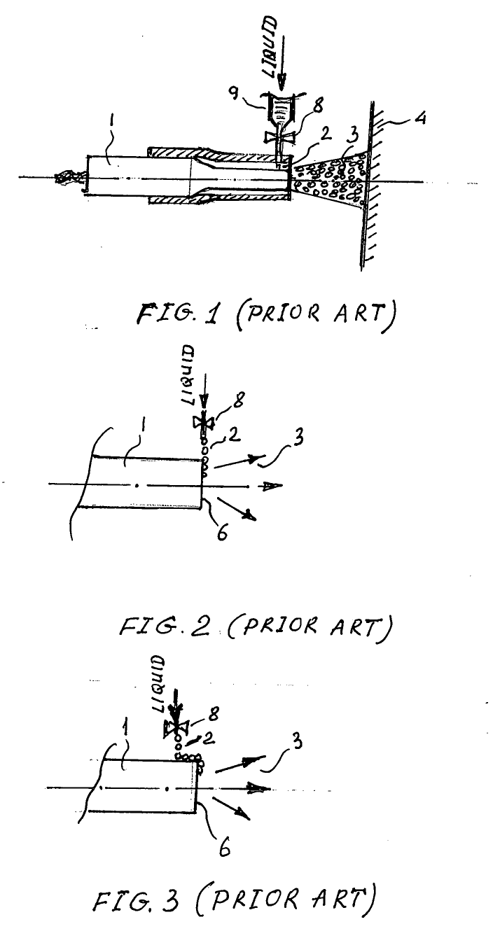

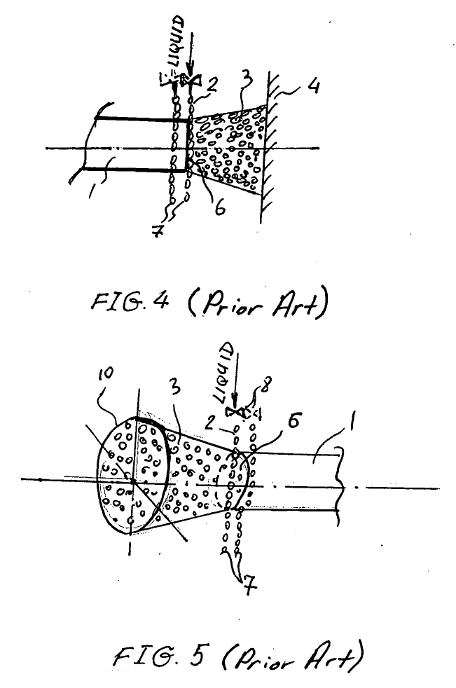

### DETAILED DESCRIPTION OF THE INVENTION

The present invention is a method and device, which uses ultrasonic energy to coat medical devices such as stents. An apparatus in accordance with the present invention may produce a highly controllable precise, fine, targeted spray. This highly controllable precise, fine, targeted spray can allow an apparatus in accordance with the present invention to coat stents without or with reduced amounts of webbing, stringing and wasting of expensive therpeutic agent than many current techniques. The following description of the present invention refers to the subject matter illustrated in the accompanying drawings. The drawings illustrate various aspects of the present inventions in the form of exemplary embodiments in which the present inventions may be practiced. These embodiments are described in sufficient detail to enable those skilled in the art to practice the present invention. Upon review of the present disclosure, it will be apparent to one skilled in the art that the various embodiments may be practiced without inclusion of some of the specific aspects. References to “an”, “one”, or “various” embodiments in this disclosure are not necessarily to the same embodiment, and such references contemplate more that one embodiment. The following detailed description is, therefore, not to be taken in a limiting sense, and the scope is defined only by the appended claims, along with the full scope of legal equivalents to which such claims are entitled.

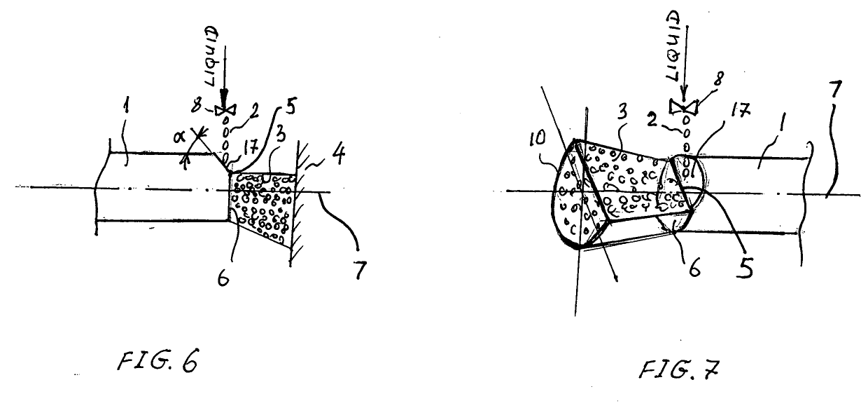

The present invention provides a novel ultrasonic tip 1 and methods for dispersing a volume of fluid to coat a stent. Embodiments of ultrasonic tips 1 in accordance with the present invention are illustrated in FIGS. 6 to 17. In accordance with the present invention, ultrasonic tip 1 includes a landing space 17 on a distal end of the ultrasonic tip 1. The landing space provides a surface on which for liquid drops 2 or liquid flow 2 may be introduced onto the ultrasonic tip 1. The ultrasonic tip 1 is typically constructed from a metal. In one aspect, the metal used can be titanium. Those skilled in the art will recognize additional materials from which the ultrasonic tips in accordance with the present invention may be manufactured. The ultrasonic tip 1 is typically connected to an apparatus (not shown) to ultrasonically vibrate the ultrasonic tip 1 as will be recognized by those skilled in the art upon review of the present disclosure.

Various configurations for landing space 17 are illustrated in FIGS. 6 to 17. In one aspect, the landing space 17 can provide substantially planar surface for introducing a liquid or therapeutic agent which avoids dripping and wasting liquid/therapeutic agent 7. In another aspect, the landing space 17 may have a curved surface. As the tip vibrates, the liquid/therapeutic agent 7 is draw from the landing space 17 where it was introduced to the radiation surface 6 of ultrasonic tip 1 from which the liquid/therapeutic agent 7 is dispersed. In one aspect, the line 5 formed by the intersection of the surface defining the landing space 17 and the surface defining the radiation surface 6 will be perpendicular to the longitudinal axis 7 of the ultrasonic tip 1 when viewed from above with reference to the orientations of the embodiments presented in FIGS. 6, 8 and 17 for example. In one aspect, landing space 17 may create a substantially flat plane in the spray pattern as is illustrated in FIGS. 6 to 17. Landing space 17 can be tilted from the horizontal axis under angle α, so that α is in the range 0<α<90°. The recommended range for the angle α is 30°<α<60°, and the preferred angle is α=45°. A syringe pump 8 may be provided for delivery of liquid 2 to the landing space 17 of ultrasonic tip 1. A syringe pump 8 can provide with precise control of the flow of liquid/therapeutic agent 7 onto an ultrasonic tip 1.

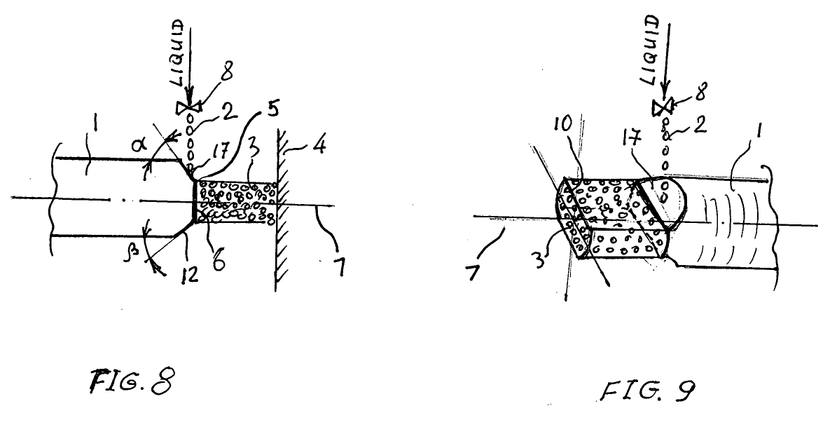

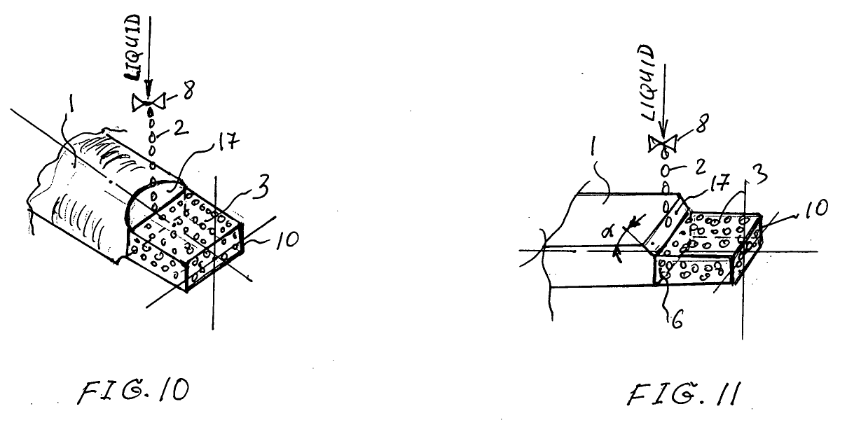

FIGS. 8 and 9 illustrate the creation of an elongated or substantially oval shaped spray pattern 10 by providing a second planar surface 12 geometrically opposite to landing space 17. Second planar surface being formed at an angle β measured from the longitudinal axis 7 which is substantially perpendicular to the radiation surface 6. This can disperse liquid/therapeutic agent 7 in a spray pattern 10 which is substantially flat on an upper side and substantially flat on a lower side. Preferably α=β. FIG. 10 shows an embodiment that creates a rectangular spray pattern 10.

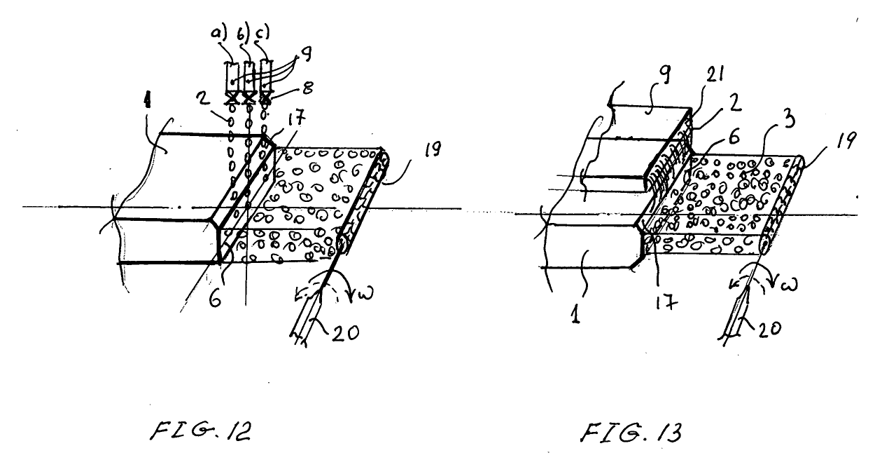

FIG. 11 illustrates a three dimensional view of an embodiment of a rectangular ultrasonic sprayer tip 1 with landing space 17 for liquid drops in one point via delivery tub/vessel 9, illustrated in FIGS. 12 and 13, in use and rectangular form of radiation surface 6 to create rectangular or flat spray 3 without dripping of portion of liquid 7 according to concept of present invention.

FIG. 12 is an illustration of a three dimensional view of an embodiment with a rectangular ultrasonic sprayer tip 1 with landing space 17 for liquid drops 2 via multiple tub/vessels 9 (a, b, c) in width of cross section in use and rectangular form of radiation surface 6 to create rectangular or flat spray 3 without dripping portion of liquid 7. FIG. 12 also shows the stent 19 spinning on a spindle or mandrel 20. The advantage or benefit of this exemplary embodiment is that by controlling the liquid flow from separate tubes, the stent surface can be coated with different or changeable thickness of coating layer along the longitudinal axis of the structure. Further, such system allows use of different therapeutic agents for coating the stents along their longitudinal axis.

FIG. 13 is a three dimensional view of an rectangular ultrasonic sprayer tip 1 with landing space 17 for liquid flow 2 in width of cross section in use and rectangular form of radiation surface 6 to create rectangular or flat spray 3 without dripping 7 according to this embodiment. Please note that liquid delivery tube/vessel's 9 cross-section 21 is rectangular as ultrasonic tip 1.

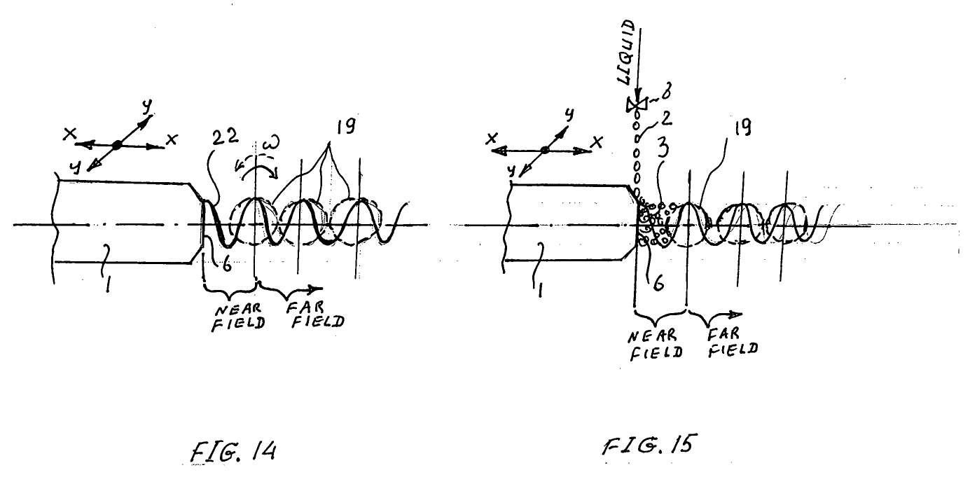

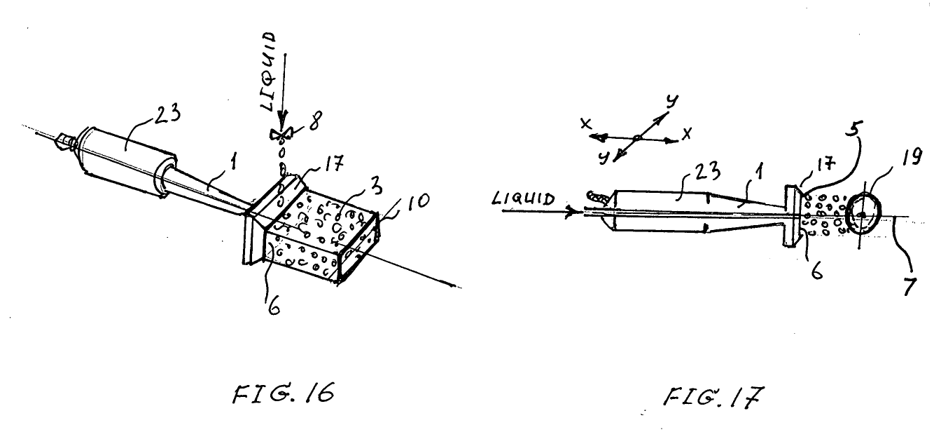

FIG. 14 is an illustration of the use of acoustic effects as part of ultrasound stent coating technique with no spray. Specifically, FIG. 14 shows a technique for improvement of the stent surface's adhesivity. Currently, one of the critical problems is getting the coating to adhere to the bare metal surface of a sent or other medical device. This embodiment provides a new approach to improve surface adhesion of bare metal stent to increase coating adherence. In this embodiment, the surface adhesivity is improved by placing the stent 19 on the front of the ultrasonic tip's 1 radiation surface 6. The ultrasonic tip 1 must be able to move toward the stent and back (x-x) and in direction of the axis of stent 19 (y-y). The reason for placing the stent in front of the radiation surface is to improve coating surface adhesion based on ionization effect of ultrasound waves in “near field” (Fresnel zone).

Clarification and description of ultrasound air ionization effect: Stable air (mainly nitrogen and oxygen) molecules are not polarized, and an ultrasound field does not affect them. Air also contains many free electrons (negative ions), which move back and forth in the ultrasound field. Overstressing of air (preferably between radiation surface and barrier) at greater than about 1 w/cm² [watts per square centimeter] can cause the free electrons in the air to attain sufficient energy to knock the free electrons from stable molecules in the air. These newly freed electrons knock off even more electrons, producing more negative and positive ions. When the oxygen molecules in the air lose electrons they become polarized positive ions. These positive ions form ozone:
O2→O+O
O+O2→O3

The fast-moving negative ions, as well as the slower heavy positive ions, bombard stent surface, eventually-destroying the insulation layers such as oxides br producing conductive “tracking” in the surface of the insulation. This produces clean surface free of oxides.

According to the theory of classical physics, free electrons are electrons not held in molecular orbit. Negative ions are free electrons. Positive ions are molecules that have lost electrons and are polarized. It is important to notice that significant ultrasonic air ionization process occurs more durable and active in-between radiation surface of the tip and barrier on front of it, such as a stent in coating process. In this condition ionization of air occurs on near field-far field interface between tip radiation surface and barrier during sonication period.

The length, L, of the near field (Fresnel zone) is equal to L=r²/λ=d²/4λ, where r is the radius and d is the diameter of the radiation surface or distal end diameter of ultrasonic tip, and λ is the ultrasound wavelength in the medium of propagation. Maximum ultrasound intensity occurs at the interface between the near field (Fresnel zone) and the far field (Fraunhofer zone). Beam divergence in the far field results in a continuous loss of ultrasound intensity with distance from the transducer. As the transducer frequency is increased, the wavelength λ decreases, so that the length of the near field increases. Ionization time can be from fraction of seconds up to minutes depending on ultrasound energy parameters and design of the ultrasound transducer/tip.

It is relevant to note that in present invention air ionization also occurs during ultrasound coating process in between spray particles in air, which also increases surface adhesion. After adhesivity improvement or surface cleaning cycle is done, without interruption of process, coating cycle must begin.

FIG. 15 illustrates the ultrasound stent coating process with spray. Stent 19 can be coated in near or far field of ultrasound field during coating process. Preferably stent must be coated at little away from near field (or in far field close to near field). Most preferably stent coating process must begin in far field, continue and finish in near field or on peak of wave amplitude. Movement of the stent back and forth in a spinning mode during coating process allows spray particles land to coating surface uniformly, in gentle manner and streamline over the surface under ultrasound pressure without stringing. At the same time ultrasound pressure wave forces, particularly ultrasound wind prevents/avoids the webbing, simply blowing up from narrow, small spaces and pushing spray particles through gaps and coating inside surface of stent walls. Further, after coating cycle and during drying cycle, as shown in FIG. 18, pressure forces including ultrasound wind dry the coating layer. Partially, wind and vaporization effect which occurs during coating acts as a drier. The thickness of the coating layer is controlled by ultrasound parameters, such as frequency/wave length, amplitude, mode of the waves (CW-continued, PW-pulse), signal form and non-ultrasound parameters like the spinning speed of stent, the distance from radiation surface, time and liquid characteristics.

Simultaneously, all three-adhesivety improvement, coating and drying cycles allows sterilization of coated stent. Sterilization occurs as a fourth cycle of the coating process due to well-known ozone bacteria and virus distruction properties.

It is important to note that the above described process can coat a portion or half a stent because the mandrel's contact area with stent on the inside cannot be coated. After reloading the stent to mandrel, the other side of the stent can be coated by repeating the process. Furthermore, the new design and construction of the holder/mandrel, the stent can be coated in one step/cycle. It is also possible to use more than one spray head with the combination of different polymer+therpeutic agent.

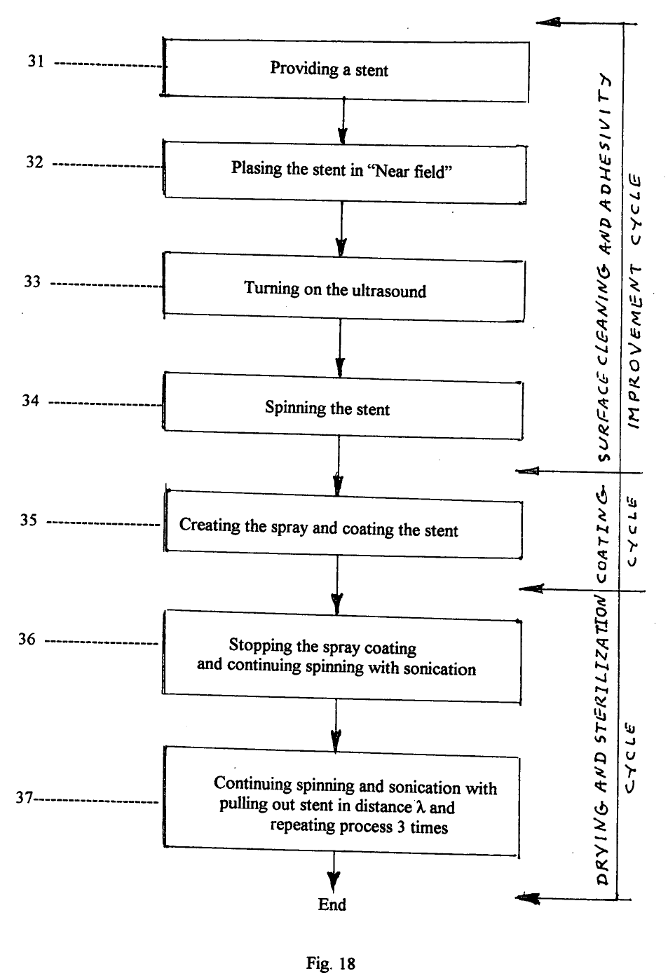

FIG. 16 is a three dimensional illustration of ultrasonic tip 1 with the specific construction of distal end for stent coating. In FIG. 16, the ultrasonic tip's distal end 6 is rectangular in order to avoid over-use or loss of expensive coating liquid such as therpeutic agent or polymer. Rectangular shape of tip's distal end matches the stent's rectangular profile in front view.

FIG. 17 is a cross sectional view of an ultrasonic sprayer 30 with the axial orifice 26 in use with the rectangular/flat spray 3 pattern 10 according to present invention.

FIG. 18 describes flow chart of an exemplary method for ultrasound stent coating process in detail and cycles in accordance with the present invention: At 31 stent is provided, meaning that stent has to be put on the mandrel. Ultrasound ionization effect in the air occurs in “near field” (Fresnel zone) and disappears in a very short time (in fraction of seconds) when radiation of ultrasound waves is off. Ozone is very unstable and dissolves with the eduction of atomic oxygen:
O3→O2+O

Because of this, all four cycles—adhesivity improvement, coating, drying and sterilization—occur without interruption of the coating cycle process.

Stent 19 in FIG. 18 must be placed in near field or preferably at the near field-far field interface during the adhesivity improvement cycle 32. Next cycle 33 turns on the ultrasound or activates the ultrasound transducer tip.

On the cycle 34 mandrel with the stent begins spinning. On the next cycle 35 the spray coating is applied to the stent. Cycle 36 includes stopping the coating and continuing spinning with the sonication process. On cycle 37, the stent is being pulled to the distance of wave length and being spun and sonicated for surface sterilization and drying purposes.

To achieve high quality and productivity method and device of present invention considers use of special hi-tech robotic system with specific Software→Hardware→Controller→Coating system with spinning mandrel (with changeable speed) and X-Y-Z direction movement.

It is important to note that all figures illustrate specific applications and embodiments of the coating process with the adhesivity improvement, coating, drying and sterilization, and are not intended to limit the scope of the present disclosure or claims to that which is presented therein. Although specific embodiments have been illustrated and described herein, it will be appreciated by those of ordinary skill in the art that any arrangement that is calculated to achieve the same purpose may be substituted for the specific embodiment shown. For example, many combinations of therpeutic agent, polymer, their temperature, cycle, sequence and times, additional gas stream (with different temperature) can be used to achieve increasing quality of coating. In various embodiments, the device can be used to coat stents with highly controllable uniformed coating layer. The modification of the device can coat the stent with changeable thickness of coating layer along the longitudinal axis of the structure.

Therefore, it is to be understood that the above description is intended to be illustrative and not restrictive. Combinations of the above embodiments and other embodiments will be apparent to those having skill in the art upon review of the present disclosure. The scope of the present invention should be determined with reference to the appended claims, along with the full scope of equivalents to which such claims are entitled.

I claim:

1. A method for coating at least a portion of one or more stents, comprising:
spinning the stent;
sonicating the stent for adhesivity improvement;
creating at least one targeted, uniform coating spray;
directing and applying a coating onto the stent;
producing at least one precise and uniform coating layer on various substrates; and
sonicating the stent after coating for sterilization.

2. The method of claim 1, further comprising placing the stent in front of the radiating surface of the ultrasonic tip without spray and sonicating for ionization of the air in order to improve surface adhesion prior to coating.

3. The method of claim 1, further comprising placing the stent in front of the radiating surface of the ultrasonic tip with spray and sonicating for ionization of the air in order to improve surface adhesion prior to coating.

4. The method of claim 2, further comprising placing the stent in front of the radiating surface of the ultrasonic tip in a near field without spray and sonicating for ionization of the air in order to improve surface adhesion prior to coating.

5. The method of claim 2, further comprising placing the stent in front of the radiating surface of the ultrasonic tip in the “far field” without spray and sonicating for ionization of the air in order to improve surface adhesion prior to coating.

6. The method of claim 2, further comprising placing the stent in front of the radiating surface of the ultrasonic tip in “near field” on peak of wave amplitude, without spray and sonicating for ionization of the air to improve surface adhesion prior to coating.

7. The method of claim 2, further comprising placing the stent in front of the radiating surface of the ultrasonic tip in “far field” on peak of wave amplitude without spray and sonicating for ionization of the air to improve surface adhesion prior to coating.

8. The method of claim 2, further comprising placing the stent in front of the radiating surface of the ultrasonic tip in “near field” between two peaks of wave amplitude, without spray and sonicating for ionization of the air in order to improve surface adhesion prior to coating.

9. The method of claim 2, further comprising placing the stent in front of the radiating surface of the ultrasonic tip in “near field” between two peaks of wave amplitude without spray and sonicating for ionization of the air in order to improve surface adhesion prior to coating.

10. The method of claim 1, further comprising placing the stent in front of the radiating surface of the ultrasonic tip in the near field-far field interface and sonicating with no spray for ionization of the air in order to improve surface adhesion prior to coating.

11. The method of claim 1, further comprising spinning the stent.

12. The method of claim 2, further comprising immobilizing the stent.

13. The method of claim 7, further comprising oscillating the distance between the radiating surface of the ultrasonic tip and the stent and spinning the stent.

14. The method of claim 8, further comprising oscillating the distance between the radiating surface of the ultrasonic tip and the stent and immobilizing the stent.

15. The method of claim 2, further comprising spraying the stent with coating material immediately after ionizing the air.

16. The method of claim 2, further comprising placing the stent in front of radiating surface of the ultrasonic tip and spraying the coating material.

17. The method of claim 2, further comprising placing the stent in front of radiating surface of the ultrasonic tip in the near field and spraying the coating material.

18. The method of claim 2, further comprising placing the stent in front of radiating surface of the ultrasonic tip in the far field and spraying the coating material.

19. The method of claim 2, further comprising placing the stent in front of the radiating surface of the ultrasonic tip in the near field-far field interface and spraying the coating material.

20. The method of claim 2, further comprising placing the stent in front of the radiating surface of the ultrasonic tip and oscillating the distance between the radiating surface of the ultrasonic tip and the stent while spraying the coating material.

21. The method of claim 16, further comprising starting the coating process in the far field and completing the coating process in the near field.

22. The method of claim 16, further comprising starting the coating process in the near field and completing the coating process in the far field.

23. The method of claim 16, further comprising starting the coating process in the far field and completing the coating process in between two peaks of wave amplitude.

24. The method of claim 16, further comprising starting coating in the near field and completing coating in the far field in between two peaks of wave amplitude.

25. The method of claim 16, further comprising beginning the coating process in the near field and completing the coating process in the near field-far field interface.

26. The method of claim 16, further comprising beginning the coating process in the near field and finishing the coating process in the far field on the peak of wave amplitude and spinning the stent.

27. The method of claim 1, further comprising sonicating the stent and spinning the stent following the completion of the coating process in order to dry the stent.

28. The method of claim 1, further comprising sonicating the stent and spinning the stent immediately following the completion of the coating process in order to sterilize the stent.

29. The method of claim 1, further comprising sonicating the stent and spinning the stent immediately following the completion of the coating process in order to simultaneously dry and sterilize the stent.

30. The method of claim 1, further comprising using different ultrasound wave amplitudes for adhesion improvement, coating, drying, and sterilization.

31. The method of claim 1, wherein the ultrasound frequency range is from 18 KHz to 60 MHz.

32. The method of claim 1, wherein the preferred range of ultrasound frequency is from 18 KHz to 200 KHz.

33. The method of claim 1, wherein the most preferable range of ultrasound frequency is from 18 KHz to 60 KHz.

34. The method of claim 1, wherein the recommended ultrasound frequency is about 50 KHz.

35. The method of claim 1, further comprising use of different ultrasound wave frequencies for adhesion improvement, coating, drying, and sterilization.

36. The method of claim 1, wherein the coating is a therapeutic agent.

37. The method of claim 1, wherein the coating is a polymer.

38. The method of claim 1, wherein coating is a mixture or combination of polymer and therapeutic agent.

39. A device for coating at least a portion of at least one stent, comprising: a. An ultrasound transducer having a tip;
b. An ultrasound transducer tip having radiating surface for emitting ultrasound energy; and
c. An ultrasound transducer tip having landing space on radiating surface of tip, providing liquid on, to produce spray without dripping.

40. A device of claim 40, wherein the ultrasound transducer frequency range is from 18 KHz to 60 MHz.

41. A device of claim 40, wherein the ultrasound transducer frequency range is from 18 KHz to 60 KHz.

42. A device of claim 40, wherein the ultrasound transducer operates at about a 50 KHz frequency.

43. A device of claim 40, wherein the ultrasonic tip's distal end amplitude range is from 3 microns to 300 microns.

44. A device of claim 40, wherein the ultrasonic tip's distal end amplitude is about 40 microns.

45. A device of claim 40, wherein the ultrasonic tip's distal end is rounded.

46. A device of claim 40, wherein the ultrasonic tip's distal end is rectangular.

47. The device of claim 40, wherein the ultrasonic tip's distal end has a landing space.

48. A device of claim 40, wherein the ultrasonic tip's distal end is a combination of different geometrical shapes and has a landing space.

49. A device of claim 40, wherein the ultrasonic tip has a central or axial orifice.

50. A device of claim 40, wherein the ultrasound transducer's driving signals are sinusoidal.

51. A device of claim 40, wherein the ultrasound transducer's driving signals are rectangular or trapezoidal.
